(19) United States
(12) Patent Application Publication
(10) Pub. No.: US 2024/0277401 A1
(43) Pub. Date: Aug. 22, 2024

(54) SURGICAL INSTRUMENT WITH CLAMP CLOSURE COMPENSATION AND RELATED METHODS

(71) Applicant: Cilag GmbH International, Zug (CH)

(72) Inventors: Dimitrios Chatzigeorgiou, Redwood City, CA (US); Yi Zhou Cheng, Stanford, CA (US); Jahanshah Fathi, New York, NY (US); Aedan M. Mangan, Cincinnati, OH (US); Mark D. Overmyer, Cincinnati, OH (US)

(21) Appl. No.: 18/170,688

(22) Filed: Feb. 17, 2023

(51) Int. Cl. A61B 18/14; A61B 34/30

(52) U.S. Cl. CPC A61B 18/1445; A61B 34/30; A61B 2034/301

(57) ABSTRACT

A surgical system with clamp closure compensation includes a surgical instrument with a shaft assembly, an end effector, and a closure assembly. The shaft assembly has an articulation section configured to articulate to a selected articulation of a plurality of articulation configurations including a straight configuration and a plurality of predetermined articulated configurations. The end effector distally extends from the articulation section and has a first jaw and a second jaw movably secured relative to each other such that the first and second jaws are configured to selectively move from an open configuration to a first closed configuration. The closure assembly is operatively connected to the end effector and configured to urge the first and second jaws from the open configuration to the first closed configuration based at least on the selected articulation of the articulation section for adjusting tissue compression to compensate for the selected articulation.

### BACKGROUND

A variety of medical instruments may be used in procedures conducted by a medical professional operator, as well as applications in robotically assisted surgeries. In the case of robotically assisted surgery, the clinician may operate a master controller to remotely control the motion of such medical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletol gloves, master manipulators, or the like), which are coupled by a servo mechanism to the medical instrument. In some scenarios, a servo motor moves a manipulator supporting the medical instrument based on the clinician's manipulation of the hand input devices. During the medical procedure, the clinician may employ, via a robotic system, a variety of medical instruments including an ultrasonic blade, a surgical stapler, a tissue grasper, a needle driver, an electrosurgical cautery probes, etc. Each of these structures performs functions for the clinician, for example, cutting tissue, coagulating tissue, holding or driving a needle, grasping a blood vessel, dissecting tissue, or cauterizing tissue.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; and U.S. Pat. No. 11,058,493, entitled “Robotic System Configured for Navigation Path Tracing,” issued Jul. 13, 2021, the disclosure of which is incorporated by reference herein, in its entirety.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a control console and a plurality of robotic arms;

FIG. 2 depicts a perspective view of an example of a robotic arm, an example of a tool drive, and a first example of a surgical instrument, each configured for use with the table-based robotic system of FIG. 1;

FIG. 3A depicts an enlarged schematic perspective view of the tool driver and the surgical instrument of FIG. 2;

FIG. 3B depicts a schematic perspective view of the tool driver similar to FIG. 3A, but with the surgical instrument removed to expose rotary drives;

FIG. 4 depicts a perspective view of a second example of a surgical instrument configured to use with the table-based robotic system of FIG. 1 as well as the robotic arm and the tool drive of FIG. 2;

FIG. 5 depicts an enlarged perspective view of a distal end portion of the surgical instrument of FIG. 4 with an end effector in an open configuration and an articulation section in a straight configuration;

FIG. 6 depicts an enlarged perspective view of the distal end portion of the surgical instrument of FIG. 4 with the end effector in the open configuration and rolled about a longitudinal axis;

FIG. 7 depicts an enlarged perspective view of the distal end portion of the surgical instrument of FIG. 4 with the end effector in the open configuration and the articulation section articulated to adjust a pitch of the end effector through a pitch plane;

FIG. 8 depicts an enlarged perspective view of the distal end portion of the surgical instrument of FIG. 4 with the end effector in the open configuration and the articulation section articulated to adjust a yaw of the end effector through a yaw plane;

FIG. 9 depicts an enlarged perspective view of the distal end portion of the surgical instrument of FIG. 4 with the end effector in a closed configuration and the yaw of the end effector adjusted through the yaw plane;

FIG. 10A depicts an enlarged perspective view of the distal end portion of the surgical instrument of FIG. 4 with a portion of the end effector in broken lines for greater clarity of a knife member in a proximal position;

FIG. 10B depicts an enlarged perspective view of the distal end portion of the surgical instrument similar to FIG. 10A, but with the knife member in a distal position;

FIG. 11 depicts an enlarged rear schematic view of a proximal end portion of the surgical instrument of FIG. 4 with a plurality of cables configured to direct manipulation of the end effector;

FIG. 12 depicts a chart of visualizing an uncompensated relationship between articulation and clamp force;

FIG. 13A depicts a side schematic view of a clamp closure system with the end effector directed to a fully open configuration;

FIG. 13B depicts the side schematic view of clamp closure system with the end effector similar to FIG. 13A, but being directed toward the closed configuration;

FIG. 13C depicts the side schematic view of the clamp closure system with the end effector similar to FIG. 13B, but in the closed configuration;

FIG. 14 depicts chart of visualizing a compensated relationship between articulation and clamp force;

FIG. 15 depicts a flowchart of a first exemplary method of clamp closure compensation for an example of an open-loop clamp closure system;

FIG. 16 depicts an enlarged rear schematic view of a proximal end portion of a third example of a surgical instrument configured to use with the table-based robotic system of FIG. 1 as well as the robotic arm and the tool drive of FIG. 2;

FIG. 17 depicts a flowchart of a second exemplary method of clamp closure compensation for an example of a closed-loop clamp closure system;

FIG. 18 depicts a flowchart of a third exemplary method of clamp closure compensation; and

FIG. 19 depicts a flowchart of a fourth exemplary method of clamp closure compensation.

### DETAILED DESCRIPTION

### I. Overview of Example of Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Example of Robotic System Table

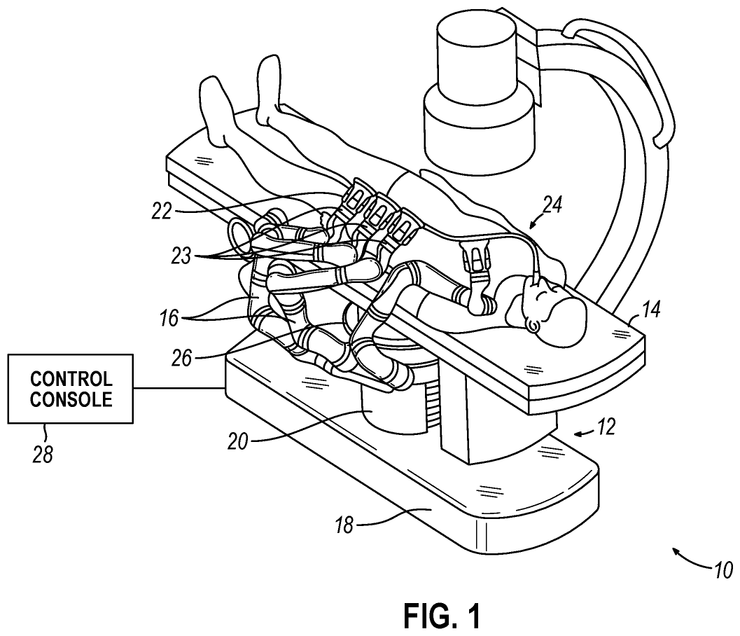

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) structurally supports platform (14), and provides a path for vertical translation of the carriages. In some versions, a table base may stow and store robotic arms (16) when not in use. Column (20) of the present example also includes a ring-shaped carriage (26), from which robotic arms (16) are based. A control console (28) is coupled with robotic surgical system (10). While four robotic arms are shown, more or fewer robotic arms are envisioned.

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable locations. Tool drivers (22) are positioned at distal ends of robotic arms (16) in the present example. Tool drivers (22) are operable to manipulate one or more surgical instruments (24), as will be described in greater detail below.

### B. Example of a Robotic Arm, Tool Drive, and Tool

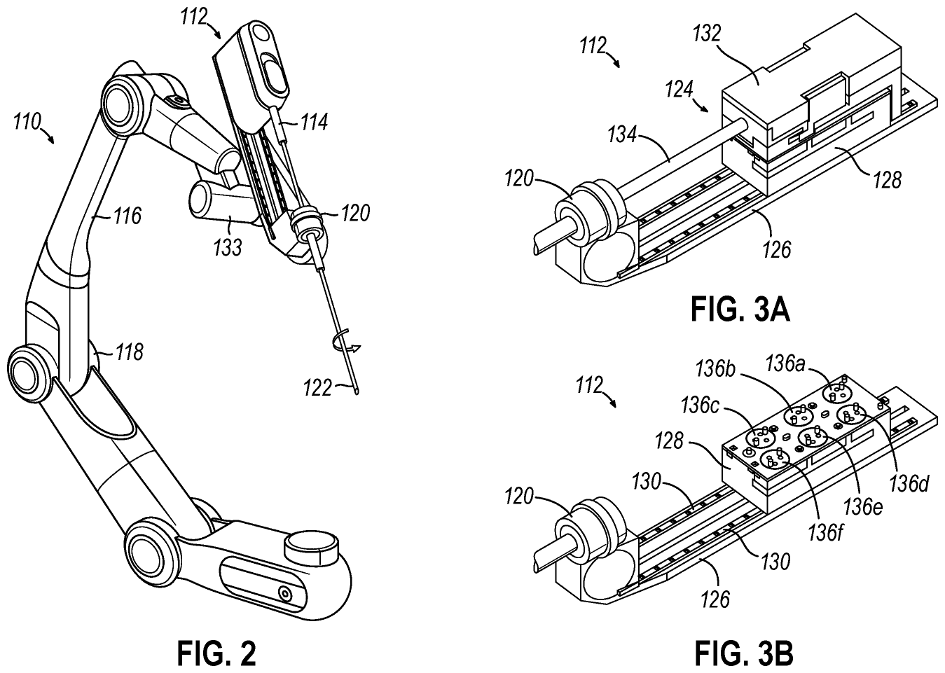

FIG. 2 shows an example of a robotic arm (110), a tool driver (112), and a surgical instrument (114), which may be incorporated into robotic surgical system (10) in place of a robotic arm (16), a tool driver (22), and a surgical instrument (24) that are shown in FIG. 1. Additional examples of robotic arms, tool drivers, and surgical instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) is attached to the distal end of robotic arm (110). Tool driver (112) includes a cannula (120) coupled to the end of tool driver (112), to receive and guide surgical instrument (114). Surgical instrument (114) may include an endoscope, a laparoscope, a stapler, graspers, an ultrasonic instrument, an RF electrosurgical instrument, or any other suitable kind of instrument. Surgical instrument (114) is inserted into the patient via cannula (120). The distal end of surgical instrument (114) includes an end effector (122). End effector (122) is configured to interact with the patient (e.g., providing visualization, stapling, grasping, ultrasonic cutting and/or sealing, electrosurgical cutting and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct a surgical instrument (114) from a desired point in space while allowing the clinician to move joints (118) into a clinically advantageous position away from the patient to create greater access, while avoiding collisions of robotic arms (110).

FIGS. 3A and 3B show tool driver (112) with and without a tool driver adapter (124), which may also be referred to as a tool base. As shown in FIGS. 3A and 3B, tool driver (112) may include a stage (126) and a carriage (128). Stage (126) includes longitudinal tracks (130). Carriage (128) is slidingly engaged with longitudinal tracks (130). Stage (126) of tool driver (112) may be configured to couple to a distal end (133) of robotic arm (110) such that articulation of robotic arm (110) positions and/or orients tool driver (112) in space. Surgical instrument (114) includes a tool driver adapter (124) at a proximal end and, as noted above, end effector (122) at a distal end. Tool driver adapter (124) includes a handle (132) and a shaft assembly (134) that extends distally from handle (132).

Carriage (128) is configured to couple with tool driver adapter (124). Carriage (128) may drive a set of articulated movements of end effector (122) and/or otherwise actuate end effector (122), such as through a cable system or wires manipulated and controlled by actuated drives. Carriage (128) may include different configurations of actuated drives, including but not limited to motorized rotary axis drives. The plurality of rotary axis drives may be arranged in any suitable manner. As shown in FIG. 3B, carriage (128) of the present example includes six rotary drives (136a-f) arranged in two rows, extending longitudinally along the base of carriage (128). Rotary drives (136a-c) are arranged in a first row that is longitudinally offset from a second row in which rotary drives (136d-f) are arranged. This staggered arrangement of rotary drives (136a-f) may reduce the width of carriage (128) and thereby provide a more compact form factor for tool driver (112). However, rotary drives (136a-f) may be provided in any other suitable arrangement. Moreover, any other suitable kind(s) of drive outputs may be provided by carriage (128), in addition to or in lieu of rotary drives (136a-f).

### II. Examples of Surgical Instruments

### A. Overview

Robotic surgical system (10) includes a limited number of robotic arms (16, 110) onto which scopes and other surgical instruments may be coupled. Given the space constraints of robotic laparoscopic instrumentation, there are competing demands for use of these limited number of robotic arms (16, 110). For example, a surgeon may not want to dedicate a robotic arm to a surgical instrument that provides only suction, only irrigation, or only electrosurgical energy. As a result, it is beneficial for a surgical instrument coupled with one of robotic arms (16, 110) to perform multiple functions during the course of a surgical procedure. Additionally, having a surgical instrument that performs multiple functions reduces or eliminates surgical time associated with exchanging a first surgical instrument with a second surgical instrument providing a different function or capability.

### B. Example of a Surgical Instrument

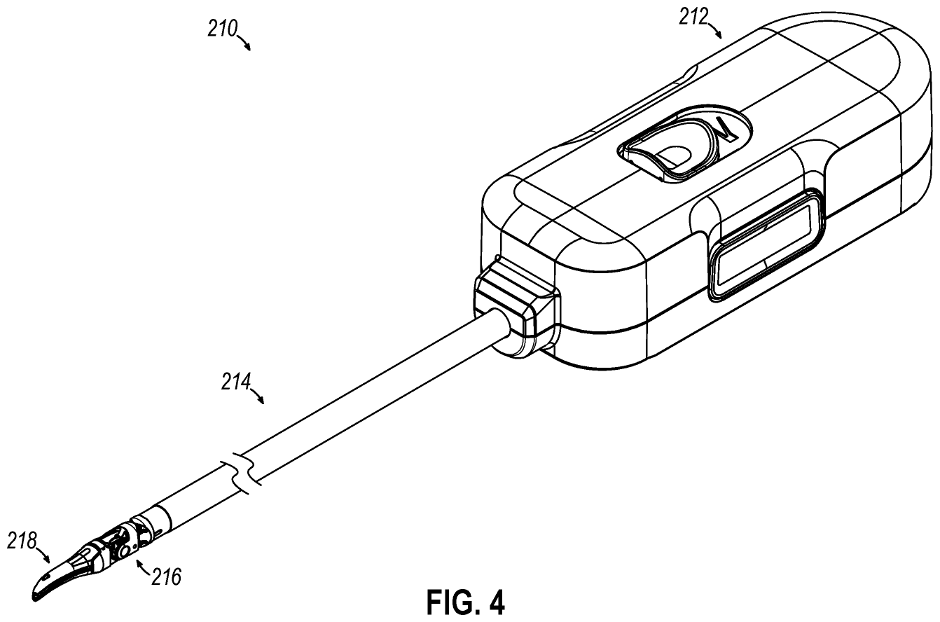

FIG. 4 shows a second example of a surgical instrument (210) configured for use with robotic surgical system (10) including robotic arms (16, 110) and tool drivers (22, 112) of FIGS. 1-2. Surgical instrument (210) may be used in place of surgical instrument (24, 114). As previously described, tool driver (112) includes stage (126) and carriage (128), with carriage (128) being configured to move relative to stage (126) to move surgical instrument (210) relative to patient (P). Surgical instrument (210) includes a body (shown as a tool drive adapter (212)), a shaft assembly (214), an articulation section (216) of shaft assembly (214), and an end effector (218).

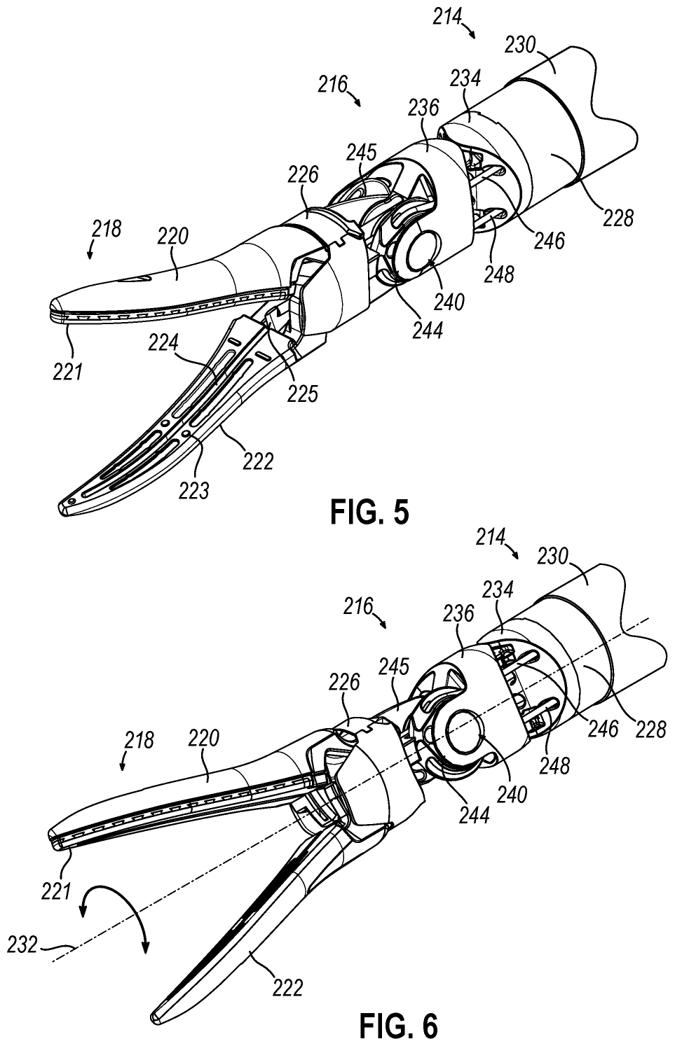

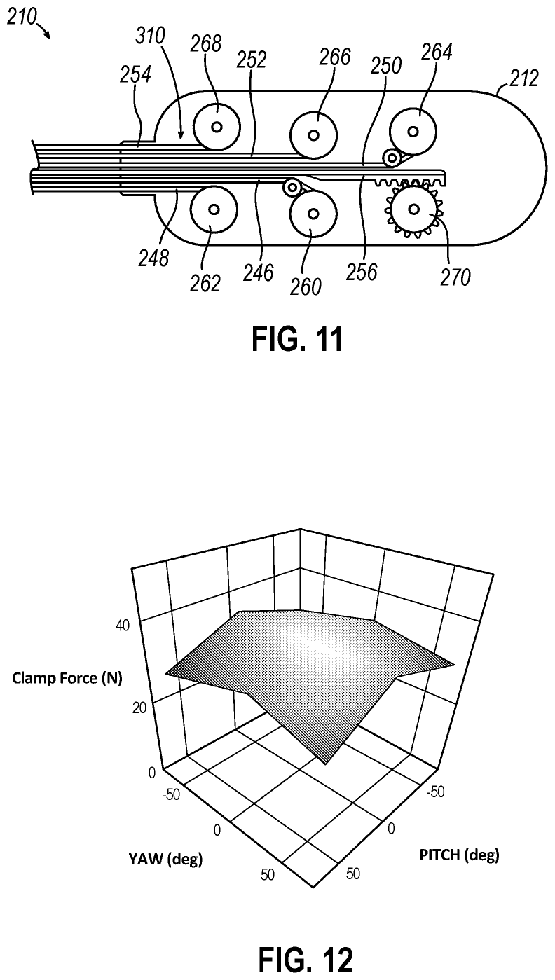

Turning to FIGS. 5-6, end effector (218) includes an upper jaw (220) having an upper electrode surface (221), a lower jaw (222) having a lower electrode surface (223), and a knife member (225) slidably disposed within a knife channel (224) cooperatively defined by upper jaw (220) and lower jaw (222). As will be described in greater detail below, end effector (218) of the current example is configured to grasp tissue with jaws (220, 222), seal tissue by applying bipolar RF energy to tissue via electrodes (221, 223), and sever tissue via knife member (225). In the current example, end effector (218) is suitably coupled to drive inputs (260, 262, 264, 266, 268, 270) (see FIG. 11) via cables and pulleys in order to suitably actuate components of end effector (218) in accordance with the description herein.

Upper jaw (220) and lower jaw (222) are pivotally coupled to each other such that jaws (220, 222) may actuate between an open configuration and a closed configuration in order to grasp tissue. In the current example, jaws (220, 222) are operatively attached to a clevis assembly (226) configured to translate to thereby pivot jaws (220, 222) between the open configuration and the closed configuration. While clevis assembly (226) is utilized in the current example, any other suitable structures may be utilized in order to drive jaws (220, 222) between the open and closed positions as would be apparent to one skilled in the art in view of the teachings herein

Articulation section (216) extends between end effector (218) and an inner shaft (228) of shaft assembly (214). Inner shaft (228) is received within an outer shaft (230) of shaft assembly (214) and configured to selectively rotate about a longitudinal axis (232) within outer shaft (230). In turn, articulation section (216) and end effector (218) are rotatably fixed relative to inner shaft (228) such that articulation section (216) and end effector (218) similarly selectively rotate with inner shaft (228). Such rotation about the longitudinal axis (232) may also be referred to herein as “roll” or “rolling” to position articulation section (216) and end effector (218) as desired for improved angles to manipulate end effector (218) as discussed below in greater detail.

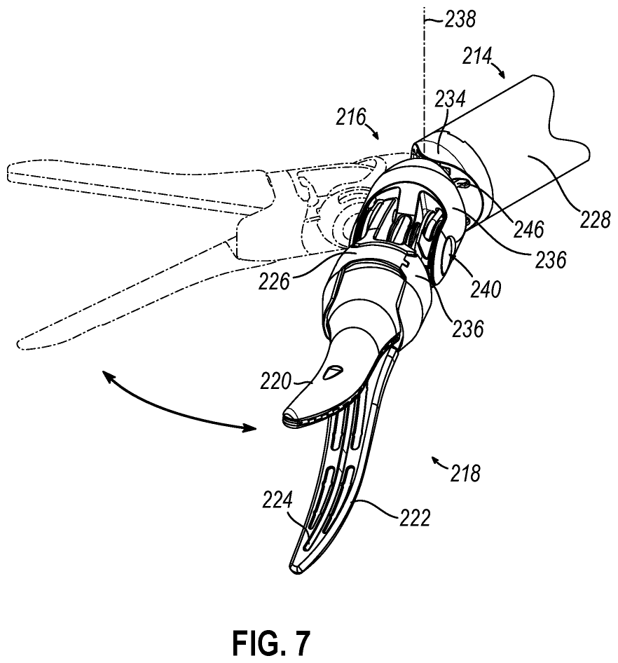

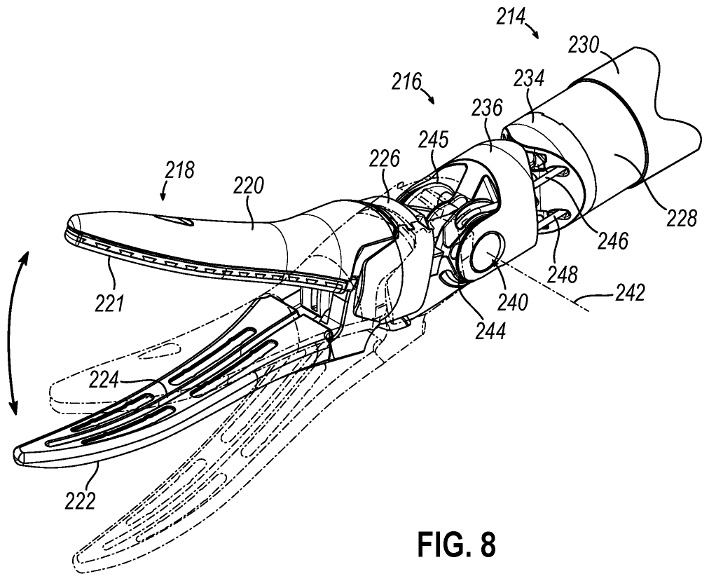

To this end, articulation section (216) is configured to articulate pitch relative to inner shaft (228) and further articulate yaw relative to inner shaft (228) to deflect end effector (218) respectively through a pitch plane as shown in FIG. 7 and a yaw plane as shown in FIG. 8. In the present example, articulation section (216) includes a proximal camming body (234) associated with a distal end of inner shaft (228) and a distal camming body (236) proximally projecting relative to end effector (218). Camming bodies (234, 236) are configured to engage each other as jaws (220, 222) deflect about a pitch axis (238), as shown in FIG. 7.

Articulation section (216) further includes a pivot coupling (240) such that jaws (220, 222) are pivotally coupled to a distal end portion of distal camming body (236). In this respect, pivot coupling (240) is configured to pivot jaws (220, 222) about a yaw axis (242). Pivot coupling (240) more particularly includes a pair of pulleys (244, 245) and a plurality of drive cables (246, 248, 250, 252) (see FIG. 11), which are configured to be collectively driven to pivot jaws (220, 222) together through yaw plane for repositioning jaws (220, 222) as desired. Pulleys (244, 245) and cables (246, 248, 250, 252) are further configured to be collectively driven to pivot jaws (220, 222) relative to each other between the open and closed configurations.

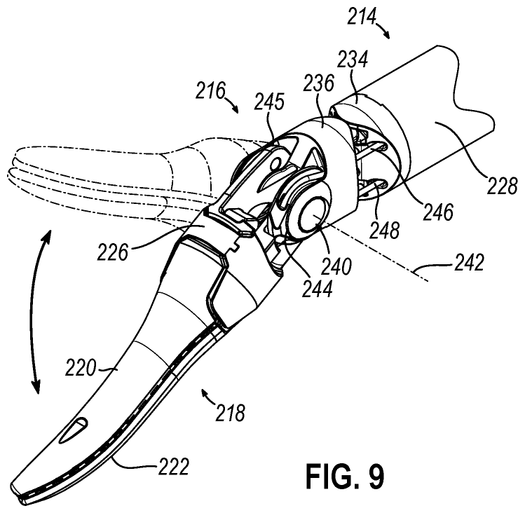

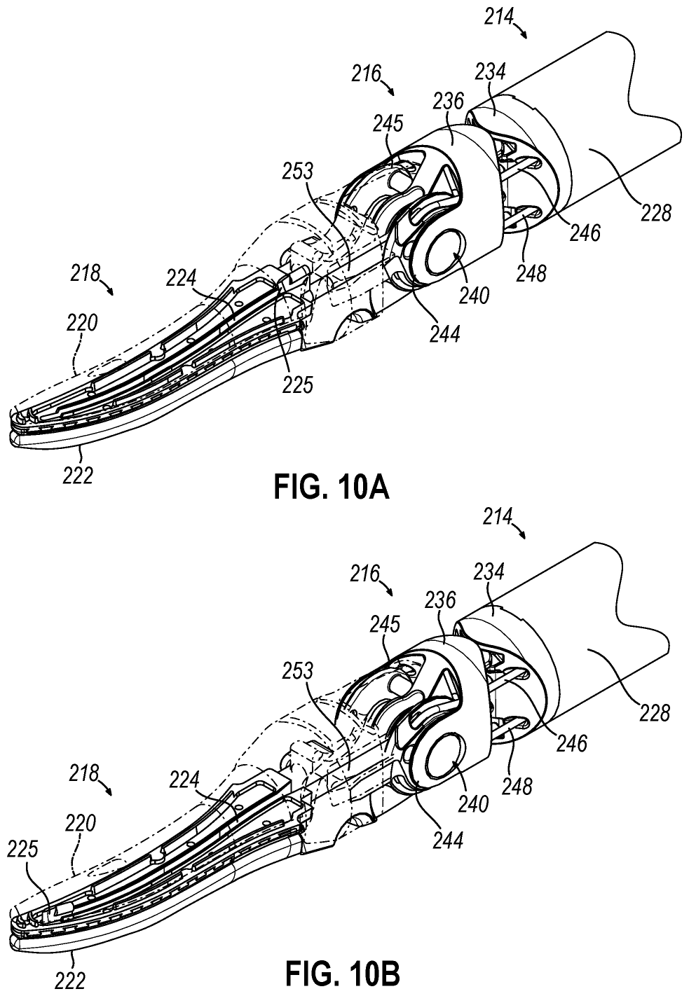

With respect to FIGS. 9-10B, while jaws (220, 222) are in the closed configuration, knife member (225) may be driven distally along a path defined by knife channel (224) from a proximal position (as illustrated in FIG. 10A) to a distal position (as illustrated in FIG. 10B) in order to sever tissue grasped by jaws (220, 222). Once knife member (225) reaches the distal position within knife channel (224) in order to suitably sever tissue, knife member (225) may then be retracted within knife channel (224) back into the proximal position. Notably, in the present example, knife member (225) distally extends from an elongate member (253), such as a nitinol tube, which is configured to translate through articulation section (216) for moving knife member (225) as discussed below in greater detail.

Electrode surfaces (221, 223) may be activated during any suitable time at which jaws (220, 222) interact with tissue in order to apply bipolar RF energy to tissue. For example, electrode surfaces (221, 223) may be activated after knife member (225) severs tissue in order to seal the recently severed tissue grasped between jaws (220, 222). As another illustrative example, electrode surfaces (221, 223) may be activated prior to knife member (225) severing tissue. As yet another illustrative example, electrode surface (221, 223) may be activated in order to cauterize tissue without cutting tissue.

In the current example, electrode surface (221) is an electrode body attached on an underside of jaw (220); while jaw (222) is formed from a suitable material in order to act as electrode surface (223). For example, jaw (222) may be formed of a metal material and be in connection with a ground wire; while electrode body forming electrode surface (221) is attached the underside of jaw (220) and in communication with a hot wire. Once suitably activated, RF energy may be transmitted between electrode surfaces (221, 223) in order to further transmit such RF energy through tissue.

Electrode surfaces (221, 223) may have any suitable configuration as would be apparent to one skilled in the art in view of the teachings herein. While in the current example, electrode surfaces (221, 223) are configured to deliver bipolar RF energy to tissue, it should be understood that end effector (218) may be configured to deliver any other suitable type of therapeutic energy to tissue as would be apparent to one skilled in the art in view of the teachings herein.

Articulation of articulation section (216) to deflect end effector (218) is directed by selectively moving associated cables (246, 248, 250, 252) as discussed briefly above, whereas roll of end effector (218) and movement of knife member (225) are directed by selectively moving associated members (254, 256). To this end, as shown in FIGS. 10B and 11, distal end portions of first upper cable (246) and first lower cable (248) attach to first pulley (244), whereas proximal end portions of first upper cable (246) and first lower cable (248) respectively attach to a first drive input (260) and a second drive input (262). Similarly, distal end portions of second upper cable (250) and second lower cable (252) attach to second pulley (245), whereas proximal end portions of second upper cable (250) and second lower cable (252) respectively attach to a third drive input (264) and a fourth drive input (266). Finally, in the present example, a fifth drive input (268) is operatively connected to inner shaft (228), articulation section (216), and end effector (218) in order to collectively direct roll of inner shaft (228), articulation section (216), and end effector (218) together, and a sixth drive input (270) is operatively connected to knife member (225) to direct movement of knife member (225) between proximal and distal positions. Such drive inputs may more particularly be referred to in one or more examples as capstans. Additional aspects of coordinating articulation of articulation section (216) as well as rolling end effector (218) and moving knife member (225) via cables (246, 248, 250, 252) are further discussed in U.S. Pat. No. 10,166,082, entitled “System and Method for Controlling a Robotic Wrist,” issued Jan. 1, 2019, the disclosure of which is incorporated by reference herein, in its entirety.

### III. Clamp Closure Compensation

As shown in the present example, knife member (225) and elongate member (253) extend through a central portion, such as along a central axis, through articulation section (216) and into end effector (218) and radially inward from cables (246, 248, 250, 252). Thus, articulation of articulation section (216) by cables (246, 248, 250, 252) similarly bends elongate member (253). This additional bend of elongate member (253) introduces an inefficiency when clamping jaws (220, 222) in the closed configuration such that greater articulation by articulation section (216) in the pitch and/or yaw planes in turn reduces clamp force between jaws (220, 222) from a predetermined, desired clamp force to a generally reduced clamp force. FIG. 12 shows one example of such reduced clamp force with greater articulation by articulation section (216) in the pitch and/or yaw planes resulting in greater reductions in clamp force. Additional positioning of end effector (218), such as by rolling end effector (218), may also affect clamp force in one or more examples.

### A. Exemplary Open-Loop Clamp Closure System

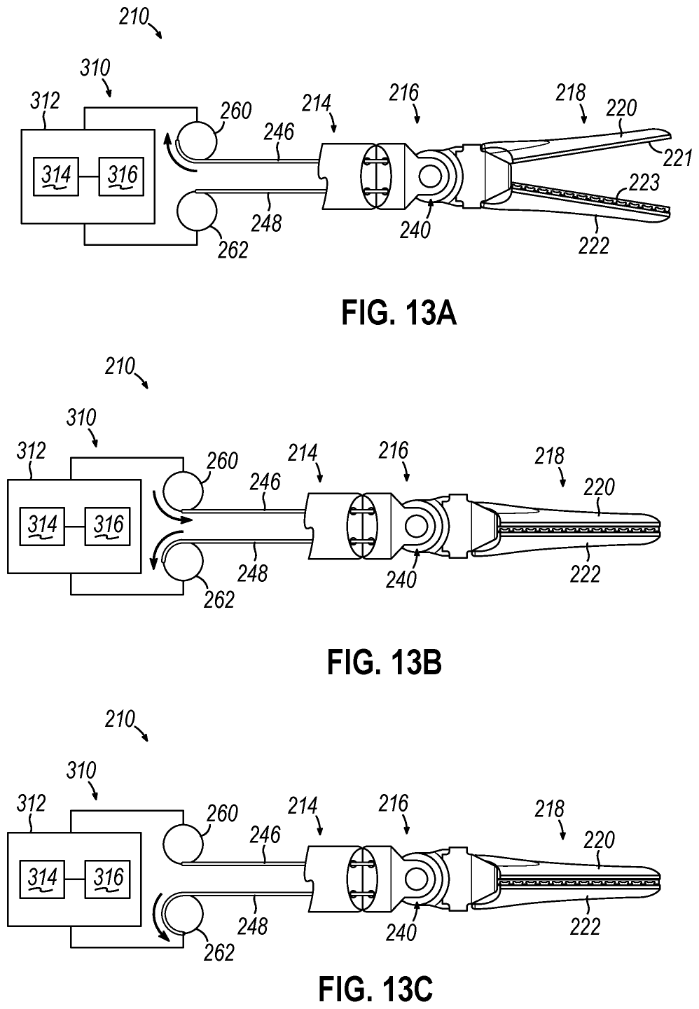

FIGS. 13A-15 show an example of an open-loop clamp closure system (310) of surgical instrument (210) including a controller (312) having a central processing unit (CPU) (314) and a memory (316) configured to compensate for reduced clamp force associated with articulation and/or roll of articulation section (216) with end effector (218). In this respect, like numbers indicate like features discussed above. Controller (312) may be incorporated into any portion of surgical system (10), such as surgical instrument (210) shown herein, and is not intended to be unnecessarily limited to incorporation into surgical instrument (210). More particularly, controller (312) is operatively connected to each of first and second drive inputs (260, 262) and configured to direct rotation of first and second drive inputs (260, 262) to in turn selectively direct draw and/or release of cables (246, 248) for moving jaws (220, 222). In this respect, FIGS. 13A-13C of the present example schematically show operation of first and second drive inputs (260, 262) and cables (246, 248) to direct jaws (220, 222) as described below, but it will be appreciated that additional inputs and cables, such as third and fourth drive inputs (264, 266) with cables (250, 252) may be similarly incorporated into open-loop clamp closure system (310). Also, articulation section (216) is generally shown in a straight configuration, but it will be appreciated that the following description of closing jaws (220, 222) similarly applies in one or more articulated configurations. The invention is thus not intended to be unnecessarily limited to the particular drive inputs (260, 262) and cables (246, 248) or a particular amount of articulation as discussed herein.

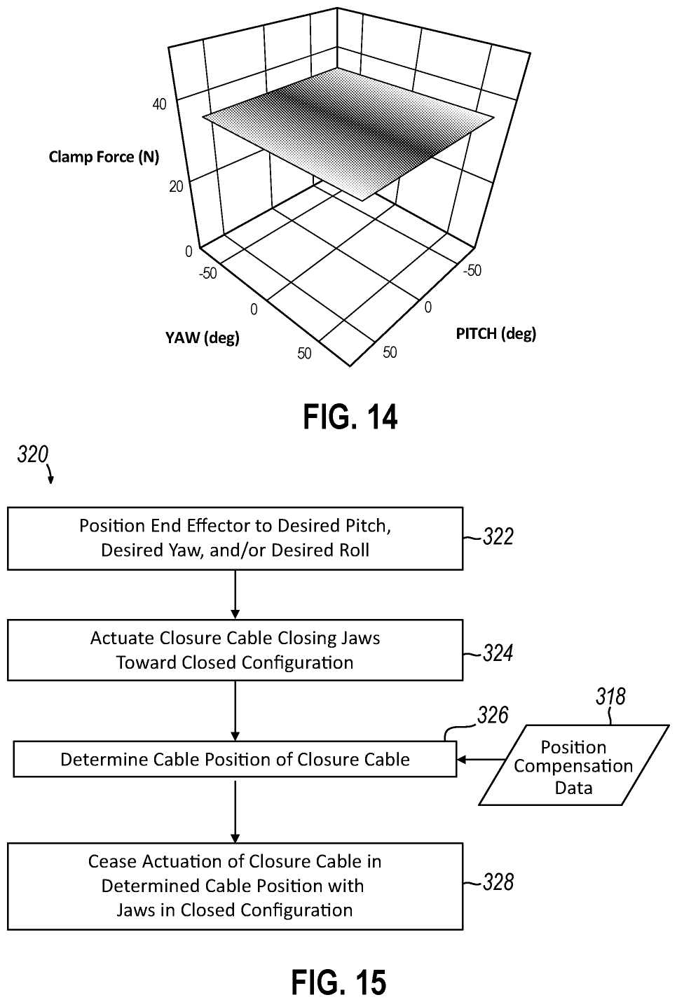

More particularly, FIG. 13A shows first drive input (260) drawing on upper cable (246) in tension to a fully open position with second drive input (262) and lower cable (248) remaining in a first position such that jaws (220, 222) are selectively directed to a fully open configuration via controller (312). To close jaws (220, 222), FIG. 13B shows first drive input (260) releasing upper cable (246) to a first position and second drive input (262) drawing on lower cable (248) to an uncompensated cable position such that jaws (220, 222) are selectively directed to an uncompensated closed configuration via controller (312) with clamping force, which, in the present example, may be reduced clamping force (see FIG. 12). In contrast, to compensate for any articulation of articulation section (216), CPU (314) of clamp closure system (310) receives position compensation data (318) from memory (316) based on articulation as set by an operator. Controller (312) thus directs second drive input (262) to further rotate and further draw on lower cable (248) a variable drive distance from uncompensated cable position to a compensated cable position in tension until jaws (220, 222) are selectively directed to a compensated closed configuration with the predetermined, desired clamp force, such as shown with respect to compensation data of the relationship between articulation and clamp force in FIG. 14.

In use, with respect to FIG. 15 and referring back to FIGS. 13B-13C, a first exemplary method of clamp closure compensation (320) for open-loop clamp closure system (310) includes the operator directing the position of end effector (218) to a desired pitch, yaw, and/or roll via controller (312) as discussed above in a step (322). In a step (324), the operator then directs jaws (220, 222) from the open configuration, such as the fully open configuration, toward the uncompensated closed configuration. Upon step (324), CPU (314) accesses memory (316) for position compensation data (318) and determines the compensated cable position of lower cable (248) that correlates to the compensated closed configuration in a step (326). Lower cable (248) thus passes through the uncompensated cable position to the compensated position so that, in turn, jaws (220, 222) move through the uncompensated closed configuration to the compensated closed configuration. Finally, controller (312) ceases actuation of jaws (220, 222) in a step (328) such that jaws (220, 222) apply the desired, predetermined clamp force.

In this respect, open-loop closure system (310) does not receive feedback of real-time clamp force, but draws upon lower cable (248) to a discrete position correlated to the compensated closed configuration stored on the memory (316). Again, while the present example depicts lower cable (248) as a closure cable for closing jaws (220, 222), it will be appreciated that movement between opened and closed configurations may be directed by more than one such cable or cables. The invention is thus not intended to be unnecessarily limited to closure via lower cable (248) as shown in the present example. Additionally, the above referenced cable positions may be taken directly from cables (246, 248, 250, 252) as applicable and/or from associated angular position of drive inputs (260, 262, 264, 266, 268, 270). Such cable positions are thus not intended to be unnecessarily limited to being determined directly from cables (246, 248, 250, 252) and, instead, may be determined by associated correlations with other features, such as associated capstans.

### B. Exemplary Closed-Loop Clamp Closure System

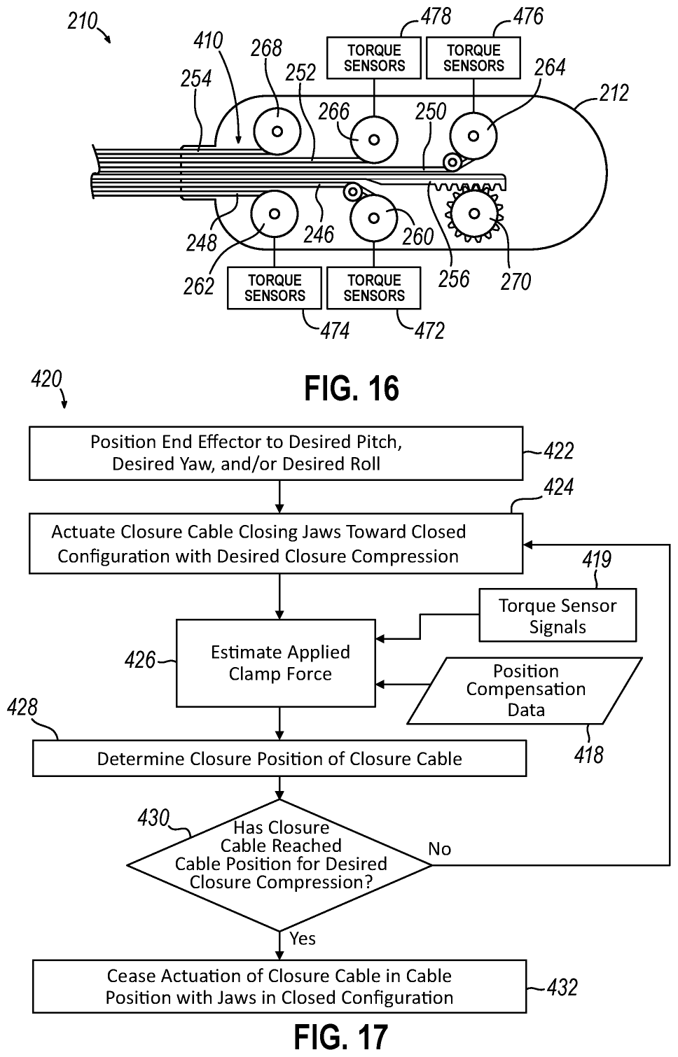

FIGS. 16-17 show an example of a closed-loop clamp closure system (410) of surgical instrument (210) including controller (312) having central processing unit (CPU) (314) and memory (316) configured to compensate for reduced clamp force associated with articulation and/or roll of articulation section (216) with end effector (218). In this respect, like numbers indicate like features discussed above with clamp closure system (310) being similar to clamp closure system (410) unless otherwise described below. For example, drive inputs (260, 262, 264, 266) are operatively connected to respective torque sensors (472, 474, 476, 478) configured to provide feedback to CPU (314) for estimating clamp force in real-time to generate the predetermined, desired clamp force with jaws (220, 222) in the compensated closed configuration. In one example, torque sensors (472, 474, 476, 478) are positioned in tool drive adapter (212). In one example, torque sensors (472, 474, 476, 478) are positioned in carriage (128) (see FIG. 3B) and incorporated into rotary drives (136a-f) (see FIG. 3B) and configured to sense torque applied at drive inputs (260, 262, 264, 266, 268, 270). Of course, any such sensor configured to detect an aspect of torque, such as force, may be alternatively positioned for providing feedback of clamp force at jaws (220, 222) such that the invention is not intended to be unnecessarily limited to torque sensors (472, 474, 476, 478) shown in the present example.

In use, with respect to FIGS. 16-17 and referring back to FIGS. 13B-13C, a second exemplary method of clamp closure compensation (420) for closed-loop clamp closure system (410) includes the operator directing the position of end effector (218) to a desired pitch, yaw, and/or roll via controller (312) as discussed above in a step (422). In a step (424), the operator then directs jaws (220, 222) from the open configuration, such as the fully open configuration, toward the uncompensated closed configuration. Upon step (424), CPU (314) estimates a real-time clamp force being applied between jaws (220, 222) in a step (466). More particularly, CPU (314) accesses memory (316) for position compensation data (418) and further receives real-time torque sensor signals (419) from applicable torque sensors, such as at least one of torque sensors (472, 474, 476, 478). Based on position compensation data (418) and real-time torque sensor signals (419), CPU (314) performs a calculation, such as by a closure model equation, to estimate the real-time clamp force being applied between jaws (220, 222). Then, in a step (428), CPU (314) determines a compensated cable position that correlates to the compensated closed configuration and is a variable drive distance further than the uncompensated cable position. In a following step (430), CPU (314) receives a real-time position of lower cable (248), compares the real-time position of lower cable (248) to the compensated cable position. In the event that the real-time position of lower cable (248) has not yet moved the variable drive distance to reach the compensated cable position such that the estimated clamp force has not yet reached the desired, predetermined clamp force, jaws (220, 222) continue closing per step (424) and CPU (314) repeats estimating an updated real-time clamp force and determining an updated compensated cable position for further refining the applied clamp force toward the desired, predetermined clamp force. However, once the real-time position of lower cable (248) reaches the compensated cable position, which may have been updated and refined based on the feedback of real-time torque sensor signals (419), estimated clamp force approximately equals the desired, predetermined clamp force within a set threshold. In turn, controller (312) ceases actuation of jaws (220, 222) in a step (432) such that jaws (220, 222) apply the desired, predetermined clamp force.

In this respect, closed-loop closure system (410) receives feedback of real-time clamp force based on torque sensor signals (419) to aid in determining the cable position that correlates to the compensated closed configuration for the desired, predetermined clamp force. In one example, another sensor may alternatively or additionally be configured to detect any aspect of real-time clamp feedback force, such as torque at torque sensors (472, 474, 476, 478), applied between jaws (220, 222) such that the invention is not intended to be unnecessarily limited to the torque sensor signals (419) as shown and described in the present example. Again, while the present example depicts lower cable (248) as a closure cable for closing jaws (220, 222), it will be appreciated that movement between opened and closed configurations may be directed by more than one such cable or cables. The invention is thus not intended to be unnecessarily limited to closure via lower cable (248) as shown in the present example. Additionally, the above referenced cable positions may be taken directly from cables (246, 248, 250, 252) as applicable and/or from associated angular position of drive inputs (260, 262, 264, 266, 268, 270). Such cable positions are thus not intended to be unnecessarily limited to being determined directly from cables (246, 248, 250, 252) and, instead, may be determined by associated correlations with other features, such as associated capstans.

### C. Additional Methods of Clamp Closure Compensation

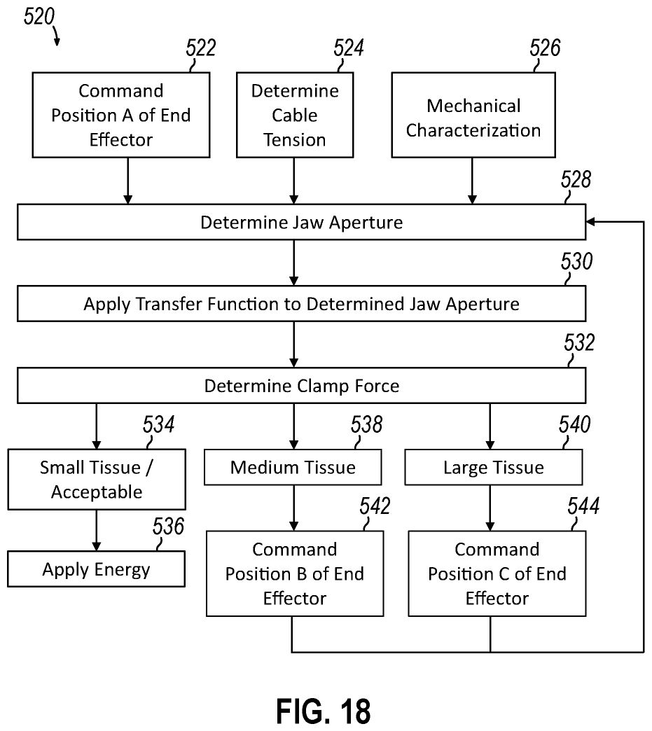

FIG. 18 shows a third exemplary method of clamp closure compensation (520) that may be incorporated, in whole or in part, into one or both of clamp closure systems (310, 410) as associated with surgical instrument (210). In the present example, with respect to FIG. 18 and referring back to FIGS. 13B-13C, the operator may direct the position of end effector (218) to a desired pitch, yaw, and/or roll via controller (312) and initiate closure of jaws (220, 222) onto a tissue to a command position A of a closed configuration as shown in a step (522). Notably, memory (316) retains predetermined reference cable tensions stored thereon that are associated with jaws (220, 222) in the command position A, such as without tissue received between jaws (220, 222). From command position A, while gripping the tissue, one or more of torque sensors (472, 474, 476, 478) (see FIG. 16) provide real-time torque sensor signals to CPU (314) to determine real-time cable tension in a step (524). Mechanical characterization unique to manufacturing variation in surgical instrument (210) may also be provided to CPU (314) in a step (526) for improved accuracy in one or more examples. Such mechanical characterization may also be stored on memory (316) for access by CPU (314).

Once in the command position A with the real-time cable tension and mechanical characterization, CPU (314) compares the real-time cable tension to the predetermined reference cable tension and then determines a jaw aperture between jaws (220, 222) in a step (528). For example, greater real-time cable tension compared relative to the predetermined reference cable tension suggests that larger tissue is compressed between jaws (220, 222) resulting in a larger jaw aperture between jaws (220, 222) than a predetermined reference jaw aperture associated with the predetermined reference cable tension. Then, in a step (530), CPU (314) applies a transfer function to this determined jaw aperture from step (528). The results of this transfer function, in a step (532), determine a clamp force applied to the tissue between jaws (220, 222) in the present command position, such as command position A.

Based on the determined clamp force, CPU (314) identifies the tissue between jaws (220, 222) as being a relatively small tissue thickness, a relatively medium tissue thickness, or a relatively large tissue thickness. In the present example, compressing relatively small tissue with command position A yields an acceptable tissue compression in a step (534) such that surgical instrument (210) applies energy to the tissue, such as RF energy, in a step (536). Alternatively, another tissue size with another desired compression may be similarly used to apply alternative energy such that the invention is not intended to be unnecessarily limited to small tissue being the acceptable size for then applying energy. In one example, should the determined tissue size be unacceptable for applying energy, such as medium or large tissue thicknesses, then CPU (314) may prevent application of energy, thus performing a check gate function on tissue between jaws (220, 222).

In one example, such as shown in the present example, CPU (314) identifies the tissue between jaws (220, 222) as being a relatively medium tissue thickness in a step (538) or a relatively large tissue thickness in a step (540). In the event of medium tissue thickness identification, CPU (314) adjust jaws (220, 222) to a command position B, such as by opening jaws (220, 222) to the command position B in a step (542). Memory (316) retains predetermined reference cable tensions stored thereon that are associated with jaws (220, 222) in the command position B, such as without tissue received between jaws (220, 222). From command position B, step (528) is again performed based on real-time cable tension and mechanical characterization to determine jaw aperture.

Alternatively, in the event of large tissue thickness identification, CPU (314) adjust jaws (220, 222) to a command position C, such as by opening jaws (220, 222) even further to the command position C in a step (544). Memory (316) retains predetermined reference cable tensions stored thereon that are associated with jaws (220, 222) in the command position C, such as without tissue received between jaws (220, 222). From command position C, step (528) is again performed based on real-time cable tension and mechanical characterization to determine jaw aperture. In this respect, feedback associated with either one of medium tissue thickness or large tissue thickness loops again through steps (528, 530, 532) until the clamp force is acceptable for the identified tissue in step (534) to apply energy per step (536).

In yet another example, rather than provide feedback through steps (538, 540, 542, 544) back to step (528), upon the identification of small, medium, or large tissue thickness in either of respective steps (534, 538, 540), energy is applied at one of three different respective levels to accommodate the small, medium, or large tissue thickness between jaws (220, 222). Notably, while the method of clamp closure compensation (520) includes the above referenced steps through to applying energy, such as in step (536), it will be appreciated that the method of clamp closure compensation (520) may be more or less steps as desired. By way of example, the method of clamp closure compensation (520) may include determining jaw aperture per step (528) without the remaining steps discussed above. By way of further example, the method of clamp closure compensation (520) may include determining clamp force per step (532) without the remaining steps discussed above. The invention is thus not intended to be unnecessarily limited to all of the above steps in the present example.

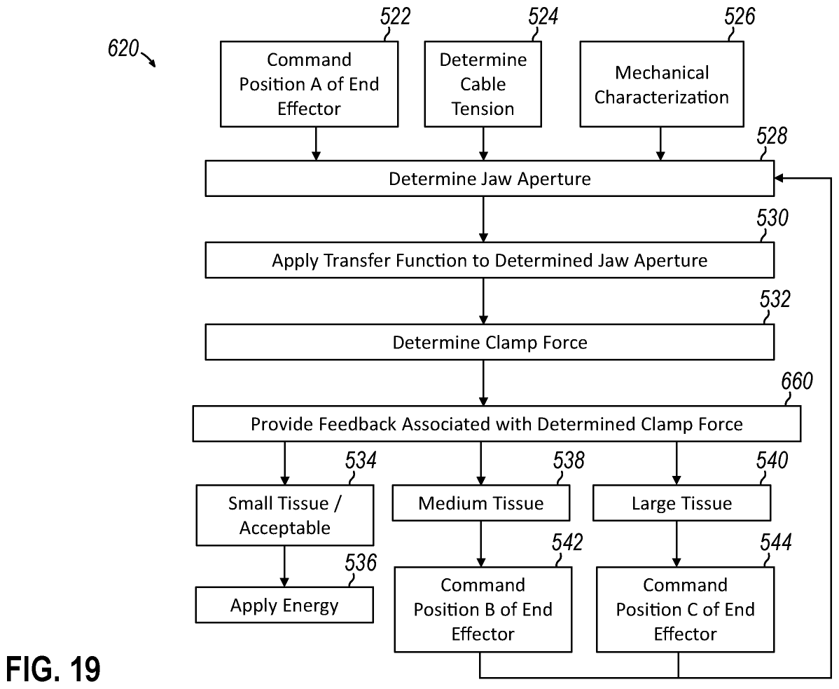

FIG. 19 shows a fourth exemplary method of clamp closure compensation (620) that may be incorporated, in whole or in part, into one or both of clamp closure systems (310, 410) as associated with surgical instrument (210). In the present example, with respect to FIG. 19 and referring back to FIGS. 13B-13C, the operator may direct the position of end effector (218) to a desired pitch, yaw, and/or roll via controller (312) and initiate closure of jaws (220, 222) onto a tissue to the command position A of the closed configuration as shown in a step (522). To this end, method of clamp closure compensation (620) is like method of clamp closure compensation (520) discussed above unless otherwise stated below. Like numbers thus indicate like features discussed above in greater detail.

More particularly, after determining clamp force in step (532), a user feedback generator of robotic surgical system (10) generates a user feedback in a step (660) configured to indicate to the operator the determined clamp force on the tissue between jaws (220, 222). In one example, the user feedback generator includes a tactile feedback generator configured to provide tactile feedback to the operator engaged with control console (28) (see FIG. 1) while directing operation of surgical instrument (210). Such tactile feedback may include, but is not limited to, vibration, increased stiffness, and/or other tactile communication to the operator and generally provides the operator with real-time feedback of tissue characteristics between jaws (220, 222) during use.

In one example, the user feedback generator includes an audible feedback generator configured to provide audible feedback to the operator engaged with control console (28) (see FIG. 1) while directing operation of surgical instrument (210). Such audible feedback may include, but is not limited to, a tone with a frequency modulated relative to clamp force, pulses of tones with the tones having a time length, pause length, and/or frequency change vibration, modulated relative to clamp force, a tone with a volume that modulates relative to clamp force, or any combination thereof. Such audible feedback generally provides the operator with real-time feedback of tissue characteristics between jaws (220, 222) during use.

In one example, based on the determined clamp force in step (532), method of clamp closure compensation (620) may further include CPU (314) performing an algorithm to limit clamp force applied to the tissue between (220, 222) regardless of the operator attempting to further compress the tissue via control console (28). Again, as with method of clamp closure compensation (520) (see FIG. 18), while method of clamp closure compensation (620) includes the above referenced steps through to applying energy, such as in step (536), it will be appreciated that the method of clamp closure compensation (620) may be more or less steps as desired. By way of example, the method of clamp closure compensation (620) may include determining j aw aperture per step (528) without the remaining steps discussed above. By way of further example, the method of clamp closure compensation (620) may include determining clamp force per step (532) without the remaining steps discussed above. The invention is thus not intended to be unnecessarily limited to all of the above steps in the present example.

While the above examples refer to clamping tissue and applying clamp force, such compensation of clamp force applied between jaws (220, 222) similarly applies to closure force, grip force, and generally any instrument configured to engage tissue at jaws. The invention is thus not intended to be unnecessarily limited to clamp force applied to tissue between jaws (220, 222) as shown in the present example.

### IV. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A robotic surgical system, comprising: (a) a surgical instrument, including: (i) a shaft assembly having an articulation section and a proximal shaft portion proximally extending from the articulation section, wherein the proximal shaft portion extends along a longitudinal axis and wherein the articulation section is configured to articulate to a selected articulation of a plurality of articulation configurations including a straight configuration and a plurality of predetermined articulated configurations, (ii) an end effector distally extending from articulation section of the shaft assembly, wherein the end effector extends along the longitudinal axis with the articulation section in the straight configuration and the end effector deflects from the longitudinal axis with the articulation section in any one of the plurality of the predetermined articulated configurations, wherein the end effector includes a first jaw and a second jaw movably secured relative to each other such that the first and second jaws are configured to selectively move from an open configuration to a first closed configuration, and (iii) a closure assembly operatively connected to the end effector and configured to urge the first and second jaws from the open configuration to the first closed configuration based at least on the selected articulation of the articulation section for adjusting tissue compression to compensate for the selected articulation.

### Example 2

The robotic surgical system of Example 1, further comprising a robotic arm, and wherein the surgical instrument is configured to connect to the robotic arm and be inserted into a patient.

### Example 3

The robotic surgical system of any one or more of Examples 1 through 2, wherein the closure assembly further includes a drive member operatively connected to at least one of the first and second jaws and configured to move to a plurality of drive positions to respectively direct the first and second jaws from the open configuration to the first closed configuration.

### Example 4

The robotic surgical system of Example 3, wherein the closure assembly includes a controller configured to receive the selected articulation and determine a variable drive distance to move the drive member based at least on the selected articulation of the plurality of articulation configurations to thereby urge the first and second jaws from the open configuration to the first closed configuration.

### Example 5

The robotic surgical system of Example 4, wherein the plurality of drive positions includes a first drive position and a second drive position, wherein the plurality of the predetermined articulated configurations includes a first articulated configuration and a second articulated configuration, wherein the controller is configured to direct the drive member to the first drive position to urge the first and second jaws to the first closed configuration based on the articulation section being in the first articulated configuration, and wherein the controller is configured to direct the drive member to the second drive position to urge the first and second jaws to the first closed configuration based on the articulation section being in the second articulated configuration.

### Example 6

The robotic surgical system of Example 5, wherein at least a portion of the drive member is configured to translate from the first drive position to the second drive position.

### Example 7

The robotic surgical system of Example 6, wherein the second articulated configuration is greater articulation than the first articulated configuration such that the at least the portion of the drive member is configured to translate through the first drive position to the second drive position to direct the first and second jaws to the first closed configuration with the articulation section in the second articulated configuration.

### Example 8

The robotic surgical system of Example 7, wherein the drive member is configured to be proximally pulled from the first drive position to the second drive position.

### Example 9

The robotic surgical system of any one or more of Examples 4 through 8, wherein the closure assembly includes a sensor portion configured to measure a closure feedback force to urge the first and second jaws from the open configuration toward the first closed configuration.

### Example 10

The robotic surgical system of Example 9, wherein the controller is configured to receive the closure feedback force and determine the variable drive distance to move the drive member based at least on the closure feedback force and the selected articulation of the plurality of articulation configurations to thereby urge the first and second jaws from the open configuration to the first closed configuration.

### Example 11

The robotic surgical system of Example 10, wherein the controller is further configured to adjust the first closed configuration of the first and second jaws to a second closed configuration of the first and second jaws based at least on the closure feedback force and the selected articulation of the plurality of articulation configurations for adjusting tissue compression to compensate for a thickness of the tissue.

### Example 12

The robotic surgical system of Example 3, wherein the closure assembly further includes a controller having a memory containing the plurality of drive positions thereon, wherein each of the plurality of drive positions correlates to the first closed configuration of the first and second jaws based on the selected articulation of the articulation section.

### Example 13

The robotic surgical system of Example 3, wherein the articulation section extends along a centerline, and wherein the drive member is offset from the centerline.

### Example 14

The robotic surgical system of any one or more of Examples 1 through 13, wherein the articulation section extends along a centerline, and wherein the end effector further includes a knife configured to move from a proximal position to a distal position, and wherein the knife is positioned on the centerline.

### Example 15

The robotic surgical system of any one or more of Examples 1 through 14, where the end effector further includes at least one RF electrode.

### Example 16

A robotic surgical system, comprising: (a) a shaft assembly having an articulation section configured to articulate to a selected articulation of a plurality of articulation configurations; (b) an end effector distally extending from articulation section of the shaft assembly and including a first jaw and a second jaw movably secured relative to each other such that the first and second jaws are configured to selectively move from an open configuration to a closed configuration; and (c) a closure assembly operatively connected to the end effector and including: (i) a drive member operatively connected to at least one of the first and second jaws and configured to move to a plurality of drive positions to respectively direct the first and second jaws from the open configuration to the closed configuration, and (ii) a controller having a memory containing the plurality of drive positions thereon, wherein each of the plurality of drive positions correlates to the closed configuration of the first and second jaws based on the selected articulation of the articulation section.

### Example 17

The robotic surgical system of Example 16, further comprising a robotic arm, and wherein the end effector is configured to connect to the robotic arm and be inserted into a patient.

### Example 18

A method compressing a tissue with a surgical instrument, comprising: (a) directing a first jaw and a second jaw of the surgical instrument toward an uncompensated closed configuration; (b) accessing position compensation data from a memory of the surgical instrument associated with a predetermined articulation of an end effector of the surgical instrument; and (c) moving the first and second jaws through the uncompensated closed configuration to a compensated closed configuration based at least on the position compensation date thereby compressing the tissue between the first and second jaws.

### Example 19

The method of Example 18, further comprising measuring a closure feedback force from compressing the tissue between the first and second jaws.

### Example 20

The method of any one or more of Examples 18 through 19, further comprising determining whether the first and second jaws have reached the compensated closed configuration.

### V. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator. Moreover, terms such as “upper” and “lower” are merely spatial terms relative to the figures and are not intended to unnecessarily limit the invention described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of instruments having RF electrodes, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical system, comprising:
(a) a surgical instrument, including:
(i) a shaft assembly having an articulation section and a proximal shaft portion proximally extending from the articulation section, wherein the proximal shaft portion extends along a longitudinal axis and wherein the articulation section is configured to articulate to a selected articulation of a plurality of articulation configurations including a straight configuration and a plurality of predetermined articulated configurations,
(ii) an end effector distally extending from articulation section of the shaft assembly, wherein the end effector extends along the longitudinal axis with the articulation section in the straight configuration and the end effector deflects from the longitudinal axis with the articulation section in any one of the plurality of the predetermined articulated configurations, wherein the end effector includes a first jaw and a second jaw movably secured relative to each other such that the first and second jaws are configured to selectively move from an open configuration to a first closed configuration, and
(iii) a closure assembly operatively connected to the end effector and configured to urge the first and second jaws from the open configuration to the first closed configuration based at least on the selected articulation of the articulation section for adjusting tissue compression to compensate for the selected articulation.

2. The surgical system of claim 1, further comprising a robotic arm, and wherein the surgical instrument is configured to connect to the robotic arm and be inserted into a patient.

3. The surgical system of claim 1, wherein the closure assembly further includes a drive member operatively connected to at least one of the first and second jaws and configured to move to a plurality of drive positions to respectively direct the first and second jaws from the open configuration to the first closed configuration.

4. The surgical system of claim 3, wherein the closure assembly includes a controller configured to receive the selected articulation and determine a variable drive distance to move the drive member based at least on the selected articulation of the plurality of articulation configurations to thereby urge the first and second jaws from the open configuration to the first closed configuration.

5. The surgical system of claim 4, wherein the plurality of drive positions includes a first drive position and a second drive position, wherein the plurality of the predetermined articulated configurations includes a first articulated configuration and a second articulated configuration, wherein the controller is configured to direct the drive member to the first drive position to urge the first and second jaws to the first closed configuration based on the articulation section being in the first articulated configuration, and wherein the controller is configured to direct the drive member to the second drive position to urge the first and second jaws to the first closed configuration based on the articulation section being in the second articulated configuration.

6. The surgical system of claim 5, wherein at least a portion of the drive member is configured to translate from the first drive position to the second drive position.

7. The surgical system of claim 6, wherein the second articulated configuration is greater articulation than the first articulated configuration such that the at least the portion of the drive member is configured to translate through the first drive position to the second drive position to direct the first and second jaws to the first closed configuration with the articulation section in the second articulated configuration.

8. The surgical system of claim 7, wherein the drive member is configured to be proximally pulled from the first drive position to the second drive position.

9. The surgical system of claim 4, wherein the closure assembly includes a sensor portion configured to measure a closure feedback force to urge the first and second jaws from the open configuration toward the first closed configuration.

10. The surgical system of claim 9, wherein the controller is configured to receive the closure feedback force and determine the variable drive distance to move the drive member based at least on the closure feedback force and the selected articulation of the plurality of articulation configurations to thereby urge the first and second jaws from the open configuration to the first closed configuration.

11. The surgical system of claim 10, wherein the controller is further configured to adjust the first closed configuration of the first and second jaws to a second closed configuration of the first and second jaws based at least on the closure feedback force and the selected articulation of the plurality of articulation configurations for adjusting tissue compression to compensate for a thickness of the tissue.

12. The surgical system of claim 3, wherein the closure assembly further includes a controller having a memory containing the plurality of drive positions thereon, wherein each of the plurality of drive positions correlates to the first closed configuration of the first and second jaws based on the selected articulation of the articulation section.

13. The surgical system of claim 3, wherein the articulation section extends along a centerline, and wherein the drive member is offset from the centerline.

14. The surgical system of claim 1, wherein the articulation section extends along a centerline, and wherein the end effector further includes a knife configured to move from a proximal position to a distal position, and wherein the knife is positioned on the centerline.

15. The surgical system of claim 1, where the end effector further includes at least one RF electrode.

16. A surgical system, comprising:
(a) a shaft assembly having an articulation section configured to articulate to a selected articulation of a plurality of articulation configurations;
(b) an end effector distally extending from articulation section of the shaft assembly and including a first jaw and a second jaw movably secured relative to each other such that the first and second jaws are configured to selectively move from an open configuration to a closed configuration; and
(c) a closure assembly operatively connected to the end effector and including:
(i) a drive member operatively connected to at least one of the first and second jaws and configured to move to a plurality of drive positions to respectively direct the first and second jaws from the open configuration to the closed configuration, and
(ii) a controller having a memory containing the plurality of drive positions thereon, wherein each of the plurality of drive positions correlates to the closed configuration of the first and second jaws based on the selected articulation of the articulation section.

17. The surgical system of claim 16, further comprising a robotic arm, and wherein the end effector is configured to connect to the robotic arm and be inserted into a patient.

18. A method compressing a tissue with a surgical instrument, comprising:
(a) directing a first jaw and a second jaw of the surgical instrument toward an uncompensated closed configuration;
(b) accessing position compensation data from a memory of the surgical instrument associated with a predetermined articulation of an end effector of the surgical instrument; and
(c) moving the first and second jaws through the uncompensated closed configuration to a compensated closed configuration based at least on the position compensation date thereby compressing the tissue between the first and second jaws.

19. The method of claim 18, further comprising measuring a closure feedback force from compressing the tissue between the first and second jaws.

20. The method of claim 18, further comprising determining whether the first and second jaws have reached the compensated closed configuration.
